(19) United States
(12) Patent Application Publication
(10) Pub. No.: US 2026/0184737 A1
(43) Pub. Date: Jul. 2, 2026

(54) METHODS OF SYNTHESIZING A TARGETING LIGAND-CONJUGATED NUCLEOTIDE PHOSPHORAMIDITE

(71) Applicant: ELI LILLY AND COMPANY, Indianapolis, IN (US)

(72) Inventors: Scott Alan FRANK, Indianapolis, IN (US); John Robert RIZZO, Indianapolis, IN (US)

(21) Appl. No.: 19/131,603

(22) PCT Filed: Nov. 22, 2023
(86) PCT No.: PCT/US2023/080894

§ 371 Date: May 21, 2025

Related U.S. Application Data
(60) Provisional application No. 63/427,490, filed on Nov. 23, 2022.

(51) Int. Cl. C07H 19/167; C07H 1/00

(52) U.S. Cl. CPC C07H 19/167; C07H 1/00

(57) ABSTRACT

Methods are disclosed for making a targeting ligand-conjugated nucleotide phosphoramidite known as adem-A-GalNAc phosphoramidite, which can be used in the synthesis of oligonucleotides such as therapeutic oligonucleotides.

### TECHNICAL FIELD

The disclosure relates generally to improved methods of making a nucleotide phosphoramidite, namely a targeting ligand-conjugated nucleotide phosphoramidite known as (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (adem-A-GalNAc phosphoramidite), which can be used in making therapeutic oligonucleotides.

### BACKGROUND

Oligonucleotides are short, polymeric sequences of nucleotides that have a wide range of applications, including use as primers, probes and therapeutics. Oligonucleotides, such as therapeutic oligonucleotides, can be chemically synthesized using a variety of known methods. Various chemical modifications can be made to one or more of the nucleotides in therapeutic oligonucleotides to introduce improved properties for in vivo administration (e.g., to stabilize an oligonucleotide against nucleases, to increase cellular uptake of the oligonucleotide, and/or to enhance other pharmacodynamic and/or pharmacokinetic properties of the oligonucleotide).

Intl. Patent Application Publication No. WO 2016/100401 describes a method of making an amidite known as adem-A-GalNAc phosphoramidite to improve therapeutic oligonucleotides for in vivo administration. The method described therein uses lengthy process steps that increase production costs and result in a less-robust and less-efficient manufacturing route, particularly when applied to large-scale, commercial manufacturing.

In view of the above, there is a need for improved methods of making adem-A-GalNAc phosphoramidite.

### BRIEF SUMMARY

The disclosure describes methods of making adem-A-GalNAc phosphoramidite. In one instance, a method of making adem-A-GalNAc phosphoramidite is provided that includes the following steps:
(1) silylating N6-benzoyladenosine to obtain N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide;
(2) protecting N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide as a methylthiomethyl ether to obtain N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide;
(3) forming a pentaacetate on (2R,3R,4R,5R)-2-amino-3,4,5,6-tetrahydroxyhexanal hydrochloride to obtain (2R,3R,4R,5R,6R)-3-acetamido-6-(acetoxymethyl)-tetrahydro-2H-pyran-2,4,5-triyl triacetate;
(4) forming a glycosidic bond on (2R,3R,4R,5R,6R)-3-acetamido-6-(acetoxymethyl)-tetrahydro-2H-pyran-2,4,5-triyl triacetate to obtain (2R,3R,4R,5R,6R)-5-acetamido-2-(acetoxymethyl)-6-(hex-5-en-1-yloxy)tetrahydro-2H-pyran-3,4-diyl diacetate;
(5) oxidizing (2R,3R,4R,5R,6R)-5-acetamido-2-(acetoxymethyl)-6-(hex-5-en-1-yloxy)tetrahydro-2H-pyran-3,4-diyl diacetate to obtain 5-(((2R,3R,4R,5R,6R)-3-acetamido-4,5-diacetoxy-6-(acetoxymethyl)-tetrahydro-2H-pyran-2-yl)oxy) pentanoic acid;
(6) amidating 5-(((2R,3R,4R,5R,6R)-3-acetamido-4,5-diacetoxy-6-(acetoxymethyl)tetrahydro-2H-pyran-2-yl)oxy) pentanoic acid to obtain (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate;
(7) substituting a sulfoxide on N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide with (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate;
(8) deprotecting (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate;
(9) tritylating (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate; and
(10) phosphorylating (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (adem-A-GalNAc phosphoramidite).

In another instance, a method of making adem-A-GalNAc phosphoramidite is provided that includes the following steps:
(1′) providing a compound comprising N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide;
(2′) providing a compound comprising (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate;
(3′) substituting a sulfoxide on N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide with (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate;
(4′) deprotecting (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate;
(5′) tritylating (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate to obtain (2S,3S,4S,5S,6S)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate; and
(6′) phosphorylating (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (adem-A-GalNAc phosphoramidite).

In some instances, N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide can be prepared by a method that includes the following steps:
(1) silylating N6-benzoyladenosine to obtain N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide; and
(2) protecting N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide as a methylthiomethyl ether to obtain N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide.

In some instances, (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate can be prepared by a method that includes the following steps:

(1) forming a pentaacetate on (2R,3R,4R,5R)-2-amino-3,4,5,6-tetrahydroxyhexanal hydrochloride to obtain (2R,3R,4R,5R,6R)-3-acetamido-6-(acetoxymethyl)-tetrahydro-2H-pyran-2,4,5-triyl triacetate;
(2) forming a glycosidic bond on (2R,3R,4R,5R,6R)-3-acetamido-6-(acetoxymethyl)-tetrahydro-2H-pyran-2,4,5-triyl triacetate to obtain (2R,3R,4R,5R,6R)-5-acetamido-2-(acetoxymethyl)-6-(hex-5-en-1-yloxy)tetrahydro-2H-pyran-3,4-diyl diacetate;
(3) oxidizing (2R,3R,4R,5R,6R)-5-acetamido-2-(acetoxymethyl)-6-(hex-5-en-1-yloxy)tetrahydro-2H-pyran-3,4-diyl diacetate to obtain 5-(((2R,3R,4R,5R,6R)-3-acetamido-4,5-diacetoxy-6-(acetoxymethyl)-tetrahydro-2H-pyran-2-yl)oxy) pentanoic acid; and
(4) amidating 5-(((2R,3R,4R,5R,6R)-3-acetamido-4,5-diacetoxy-6-(acetoxymethyl)tetrahydro-2H-pyran-2-yl)oxy) pentanoic acid to obtain (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate.

An advantage of the methods herein is that the materials used are available at commercial scale.

An advantage of the methods herein is that they are less-resource intensive, cheaper, and/or facilitates more efficient production of adem-A-GalNAc phosphoramidite.

An advantage of the methods herein is that they are based upon a convergent approach for making adem-A-GalNAc phosphoramidite, which reduces the number of steps required to produce adem-A-GalNAc phosphoramidite and which improves efficiency as compared to a linear approach.

### BRIEF DESCRIPTION OF THE DRAWINGS

The advantages, effects, features, and objects other than those set forth above will become more readily apparent when consideration is given to the detailed description below. Such detailed description refers to the following drawing(s), where:

FIG. 1A and FIG. 1B depict an exemplary scheme for making adem-A-GalNAc phosphoramidite.

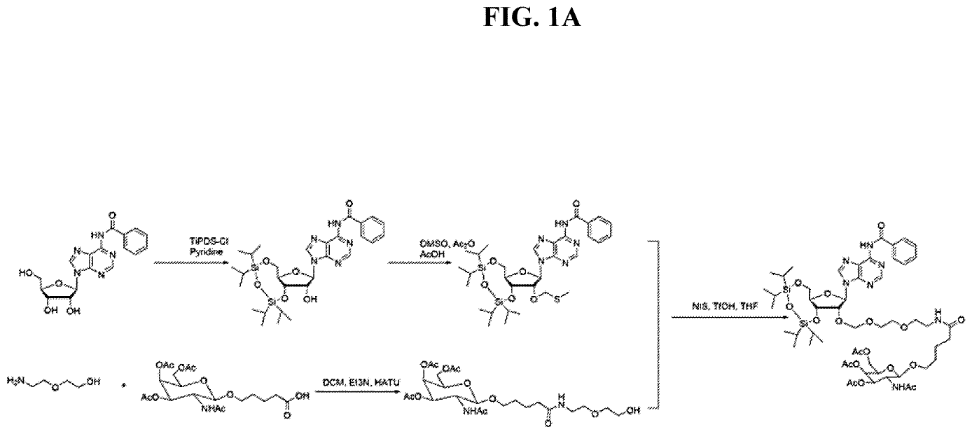

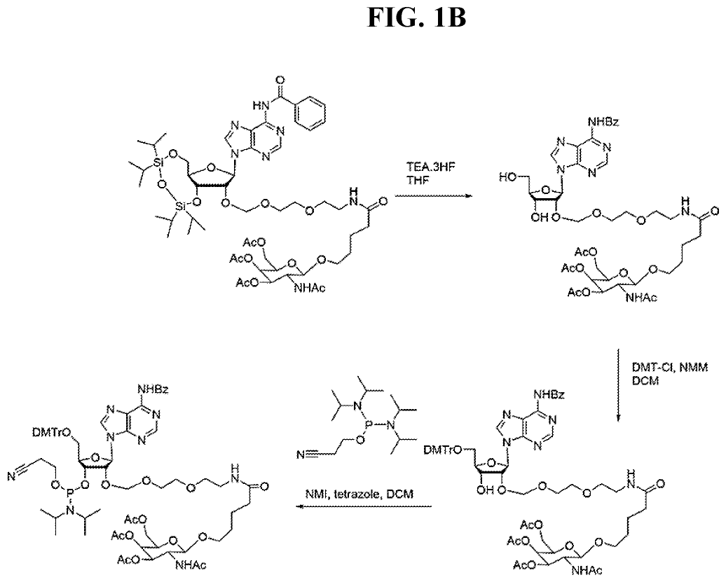

### DETAILED DESCRIPTION

### Overview

Chemical modifications can be introduced into a therapeutic oligonucleotide to confer properties that may be desired under specific conditions, such as conditions experienced following its in vivo administration. These modifications can be introduced in the base, sugar and/or phosphate group of one or more nucleotides of the oligonucleotide. Such modifications include those designed, for example: (i) to stabilize the oligonucleotide against nucleases or other enzymes that degrade or interfere with the structure or activity of the oligonucleotide, (ii) to increase cellular uptake of the oligonucleotide, and/or (iii) to improve the pharmacokinetic properties of the oligonucleotide.

For example, a therapeutic oligonucleotide can include a targeting ligand at a 5′-terminus or a 3′-terminus, or on an internal nucleotide, to selectively target the oligonucleotide to a desired cell, tissue and/or organ. One such targeting ligand is N-acetylgalactosamine (GalNAc), which can be incorporated into a therapeutic oligonucleotide, including in the form of adem-A-GalNAc phosphoramidite. See, Intl. Patent Application Publication No. WO 2016/100401.

### Abbreviations and Definitions

Unless defined otherwise, all technical and scientific terms used herein have the same meaning as commonly understood by one of skill in the art to which the disclosure pertains. Although any methods and materials similar to or equivalent to those described herein can be used in the practice or testing of the methods, the exemplary methods and materials are described herein.

Additionally, reference to an element by the indefinite article “a” or “an” does not exclude the possibility that more than one element is present, unless the context clearly requires that there be one and only one element. The indefinite article “a” or “an” thus usually means “at least one.”

Moreover, use of “including,” as well as other forms, such as “include,” “includes” and “included” is not limiting.

Certain abbreviations used herein are as follows:

“AcOH” refers to acetic acid (C₂H₄O₂); “Ac₂O” refers to acetic anhydride (C₄H₆O₃); “CDI” refers to 1,1′-carbonyldiimidazole; “DCM” refers to dichloromethane (CH₂Cl₂); “DIPDS” refers to 1,3-dichloro-1,1,3,3-tetraisopropyldisiloxane; “DMAP” refers to 4-dimethylaminopyridine (C₇H₁₀N₂); “DMSO” refers to dimethyl sulfoxide (C₂H₆OS); “DMSO-do” refers to deuterated dimethyl sulfoxide (C₂D₆OS); “DMTr-Cl” refers to 4,4′-dimethoxytriphenylmethyl chloride (C₂₁H₁₉ClO₂); “DNA” refers to deoxyribonucleic acid; “EA” refers to ethyl acetate (C₄H₈O₂); “ES-MS” refers to electrospray mass spectrometry; “eq” refers to equivalent(s); “GalNAc” refers to N-acetylgalactosamine; “HPLC” refers to high-performance liquid chromatography; “hr” refers to hour(s); “Me” refers to methyl (—CH₃); “MeOH” refers to methanol (CH₄O); “min” refers to minute(s); “MTBE” refers to methyl tertiary-butyl ether (C₅H₁₂O); “m/z” refers to mass-to-charge ratio; “NIS” refers to N-iodosuccinimide (C₄H₄INO₂); “NMI” refers to 1-methylimidazole; “NMM” refers to N-methylmorpholine; “RNA” refers to ribonucleic acid; “TEA” refers to triethylamine (C₆H₁₅N); “TfOH” refers to trifluoromethanesulfonic acid (CF₃SO₃H); “THF” refers to tetrahydrofuran (C₄H₈O); and “V” refers to volume(s).

Certain definitions used herein are as follows:

As used herein, “about” means within a statistically meaningful range of a value or values such as, for example, a stated concentration, length, molecular weight, pH, sequence similarity, time frame, temperature, volume, etc. Such a value or range can be within 20%, within 15%, within 10%, or more typically within 5% of a given value or range. Alternatively, and with respect to biological systems or processes “about” can mean within an order of magnitude such as, for example, within five-fold or more typically within two-fold of a given value. The allowable variation encompassed by “about” will depend upon the system under study, and can be readily appreciated by one of skill in the art.

As used herein, “modified nucleobase” means a nucleobase including a modified purine or pyrimidine base (e.g., adenine (A), guanine (G), cytosine (C), thymine (T) and uracil (U)). Examples of modified nucleobases include, but are not limited to, diaminopurine and its derivatives, alkylated purines or pyrimidines, acylated purines or pyrimidines thiolated purines or pyrimidines, and the like. Other modified nucleobases include analogs of purines and pyrimidines including, but not limited to, 1-methyladenine, 2-methyladenine, N6-methyladenine, N6-isopentyladenine, 2-methylthio-N6-isopentyladenine, N,N-dimethyladenine, 8-bromoadenine, 2-thiocytosine, 3-methycytosine, 5-methycytosine, 5-ethycytosine, 4-acetycytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl) uracil, 5-(methylaminomethyl) uracil, 5-(carboxymethylaminomethyl)-uracil, 2-thiouracil, 5-methyl-2-thiouracil, 5-(2-bromovinyl) uracil, uracil-5-oxyacetic acid, uracil-5-oxyacetic acid methylester, pseudouracil, 1-methylpseudouracil, queosine, hypoxanthine, xanthine, 2-aminopurine, 6-hydroxyarninopurine, nitropyrrolyl, nitroindolyl and difluorotolyl, 6-thiopurine and 2,6-diaminopurine nitropyrrolyl, nitroindolyl and difluorotolyl. Alternatively, a modified nucleobase may not contain a nitrogen atom (i.e., a universal base). See also, Intl. Patent Application Publication No. WO 2003/040395. Alternatively, the modified nucleobase is abasic (i.e., does not include a nucleobase).

As used herein, “modified nucleoside” means a nucleoside including a modified or universal nucleobase and/or a modified sugar. The modified or universal nucleobase (also referred to herein as a base analog) can be located at the 1′-position of the sugar moiety and refer to nucleobases other than adenine (A), guanine (G), cytosine (C), thymine (T) and uracil (U) at the 1′-position. In some instances, the modified nucleotide does not contain a nucleobase (abasic). The modified sugar (also referred to herein as a sugar analog) includes modified deoxyribose or ribose moieties (e.g., where the modification occurs at the 2′-, 3′-, 4′- or 5′-carbon position of the sugar). The modified sugar may also include non-natural alternative carbon structures such as those present in bridged nucleic acids (“BNA”), locked nucleic acids (“LNA”) and/or unlocked nucleic acid (“UNA”).

As used herein, “modified nucleotide” means a nucleotide including a modified or universal nucleobase as described above, a modified sugar as described above, and/or a modified phosphate or phosphate group. The modified phosphate can be a modification of the phosphate or phosphate group that does not occur in natural nucleotides and includes non-naturally occurring phosphate mimics as are known in the art. Modified phosphate or phosphate groups also include non-naturally occurring internucleotide linking groups, including both phosphorous-containing linking groups and non-phosphorous-containing linking groups as are known in the art. Suitable modified or universal nucleobases, modified sugars, and modified phosphates or phosphate groups are described herein.

As used herein, “nucleobase” means a heterocyclic nitrogenous base capable of forming Watson-Crick-type hydrogen bonds and stacking interactions in pairing with a complementary nucleobase or nucleobase analog (i.e., derivatives of nucleobases) when that nucleobase is incorporated into a polymeric structure. The natural heterocyclic nitrogenous bases include purines and pyrimidines such as adenine (A), guanine (G), cytosine (C), thymine (T) and uracil (U).

As used herein, “nucleoside” means a heterocyclic nitrogenous base in N-glycosidic linkage with a sugar moiety (e.g., deoxyribose, ribose or analog thereof). The natural heterocyclic nitrogenous bases include adenine (A), guanine (G), cytosine (C), thymine (T) and uracil (U).

As used herein, “nucleoside phosphoramidite” means a derivative of a natural or synthetic nucleoside in which reactive hydroxy and exocyclic amino groups present in natural or synthetic nucleosides are appropriately protected to prevent undesired side reactions during the synthesis of nucleic acids.

As used herein, “nucleotide” means an organic compound having a nucleoside (a nucleobase such as, for example, adenine, cytosine, guanine, thymine, or uracil; and a pentose sugar such as, for example, ribose or 2′-deoxyribose) and a phosphate group. A “nucleotide” can serve as a monomeric unit of nucleic acid polymers such as DNA and ribonucleic acid (RNA).

As used herein, “nucleotide phosphoramidite” means a derivative of a natural or synthetic nucleotide in which reactive hydroxy and exocyclic amino groups present in natural or synthetic nucleotides are appropriately protected to prevent undesired side reactions during nucleic acid synthesis.

As used herein, “oligonucleotide” means a short nucleic acid (e.g., less than about 100 nucleotides in length) of ribonucleotides, deoxyribonucleotides or a combination thereof. An oligonucleotide may be single-stranded (ss) or double-stranded (ds). An oligonucleotide may or may not have duplex regions. As a set of non-limiting examples, the oligonucleotide may be, but is not limited to, a small interfering RNA (siRNA), microRNA (miRNA), short hairpin RNA (shRNA), dicer substrate interfering RNA (dsiRNA), antisense oligonucleotide (ASO), short siRNA or ss siRNA.

As used herein, “phosphoramidite” means a nitrogen-containing, trivalent phosphorus derivative that can have a formula of (RO)₂PNR₂.

As used herein, “protecting group” means a group that reversibly renders unreactive a functional group under certain conditions of a desired reaction. After the desired reaction, the protecting group can be removed to deprotect the protected functional group. The protecting group should be removable under conditions that do not degrade a substantial proportion of the molecule (i.e., an oligonucleotide) being synthesized.

As used herein, “ribonucleotide” means a natural or modified nucleotide that has a hydroxyl group at the 2′-position of the sugar moiety.

As used herein, “targeting ligand” means a chemical moiety that facilitates entry of an oligonucleotide such as an RNAi agent into a cell. It can be a compound (e.g., an amino sugar, carbohydrate, cholesterol, lipid or polypeptide) that selectively binds to a cognate compound (e.g., a receptor) of a tissue or cell of interest and that is conjugatable to another substance for targeting another substance to the tissue or cell of interest. For example, a targeting ligand may be conjugated to an oligonucleotide for purposes of targeting it to a specific tissue or cell of interest. A targeting ligand can selectively bind to a cell surface receptor. Accordingly, a targeting ligand, when conjugated to an oligonucleotide, facilitates delivery of the oligonucleotide into a particular cell through selective binding to a receptor expressed on the surface of the cell and endosomal internalization by the cell of the complex comprising the oligonucleotide, targeting ligand, and receptor. Moreover, a targeting ligand can be conjugated to an oligonucleotide via a linker that is cleaved following or during cellular internalization such that the oligonucleotide is released from the targeting ligand in the cell.

### Compositions

adem-A-GalNAc Phosphoramidite:

The structure of adem-A-GalNAc phosphoramidite is as follows:

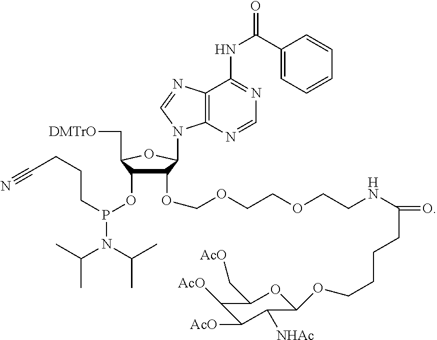

See, Intl. Patent Application Publication No. WO 2016/100401.
adem-A-GalNAc-Modified Oligonucleotides:

adem-A-GalNAc phosphoramidite can be incorporated into an oligonucleotide, such as a therapeutic oligonucleotide. In some instances, adem-A-GalNAc phosphoramidite can be incorporated at the 3′-terminus of the oligonucleotide. In other instances, adem-A-GalNAc phosphoramidite can be incorporated at the 5′-terminus of the oligonucleotide. In yet other instances, adem-A-GalNAc phosphoramidite can be incorporated at both of the 5′-terminus and 3′ terminus of the oligonucleotide. In yet other instances, adem-A-GalNAc phosphoramidite can be incorporated at one or more internal positions of the oligonucleotide. See, e.g., Intl. Patent Application Publication Nos. WO 2016/100401, WO 2021/188795, WO 2022/032288 and WO 2022/221430.

Oligonucleotides (e.g., a ds oligonucleotide such as a targeting ligand-conjugated oligonucleotide) can be made using methods and/or techniques known to one of skill in the art such as, for example, conventional nucleic acid solid-phase synthesis. The nucleotides of the oligonucleotides can be assembled on a suitable nucleic acid synthesizer utilizing standard nucleotide or nucleoside precursors (e.g., phosphoramidites). Automated nucleic acid synthesizers, including DNA/RNA synthesizers, are commercially available from, for example, Applied Biosystems (Foster City, CA), BioAutomation (Irving, TX) and GE Healthcare Life Sciences (Pittsburgh, PA).

In some instances, oligonucleotide synthesis steps can be performed in an alternate order to give the desired compounds. Other synthetic chemistry transformations, protecting groups (e.g., for hydroxyl, amino, etc. present on the bases) and protecting group methodologies (protection and deprotection) useful in synthesizing the oligonucleotides are known in the art and are described in, for example, Larock, “Comprehensive Organic Transformations,” VCH Publishers (1989); Greene & Wuts, “Protective Groups in Organic Synthesis,” 2^{nd} Ed., John Wiley & Sons (1991); Fieser & Fieser, “Fieser and Fieser's Reagents for Organic Synthesis,” John Wiley & Sons (1994); and Paquette, ed., “Encyclopedia of Reagents for Organic Synthesis,” John Wiley & Sons (1995).

### Pharmaceutical Compositions:

adem-A-GalNAc-modified oligonucleotides (or a pharmaceutically acceptable salt thereof such as, for example, trifluoracetate salts, acetate salts or hydrochloride salts) can be incorporated into a pharmaceutical composition, which includes an effective amount of adem-A-GalNAc-containing oligonucleotides and a pharmaceutically acceptable carrier, delivery agent or excipient. See, e.g., Intl. Patent Application Publication Nos. WO 2016/100401, WO 2021/188795, WO 2022/032288 and WO 2022/221430.

Various formulations have been developed to facilitate oligonucleotide use. In some instances, oligonucleotides can be delivered to an individual or a cellular environment using a formulation that minimizes degradation, facilitates delivery and/or uptake, or provides another beneficial property to the oligonucleotides in the formulation. In some instances, the oligonucleotides can be formulated in buffer solutions such as phosphate buffered saline solutions, liposomes, micellar structures and capsids.

In some instances, oligonucleotides can be reacted with an inorganic and organic acid/base to form pharmaceutically acceptable acid/base addition salts. In some instances, forming a pharmaceutically acceptable acid/base addition salt improves the in vivo compatibility and/or effectiveness of the oligonucleotide. Pharmaceutically acceptable salts and common methodologies for preparing them are well known in the art (see, e.g., Stahl et al., “Handbook of Pharmaceutical Salts: Properties, Selection and Use,” 2nd Revised Edition (Wiley-VCH, 2011)). Pharmaceutically acceptable salts for use herein include sodium, trifluoroacetate, hydrochloride and acetate salts.

In some instances, pharmaceutical compositions can be formulated to be compatible with an intended route of administration. Routes of administration include, but are not limited to, parenteral (e.g., intravenous, intramuscular, intraperitoneal, intradermal, and subcutaneous), oral (e.g., inhalation), transdermal (e.g., topical), transmucosal and rectal administration.

Moreover, factors such as solubility, bioavailability, biological half-life, route of administration, product shelf life, as well as other pharmacological considerations can be contemplated by one of skill in the art of preparing such pharmaceutical formulations, and as such, a variety of dosages and treatment regimens may be desirable.

In some instances, the pharmaceutical composition can include one or more additional therapeutic agents.

### Methods

### Methods of Making Adem-A-GalNAc Phosphoramidite:

The methods of making adem-A-GalNAc phosphoramidite or a salt thereof can include the steps described herein, which may be, but not necessarily, carried out in the sequence as described. Other sequences, however, also are conceivable. Furthermore, individual or multiple steps may be carried out either in parallel and/or overlapping in time and/or individually or in multiply repeated steps. The products of each step below can be recovered by conventional methods, including chromatography, crystallization, evaporation, extraction, filtration, precipitation and trituration.

In one instance, adem-A-GalNAc phosphoramidite can be prepared according to the method below, which can include the following steps:
(1) silylating N6-benzoyladenosine,

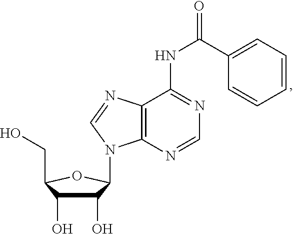

to obtain N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide,

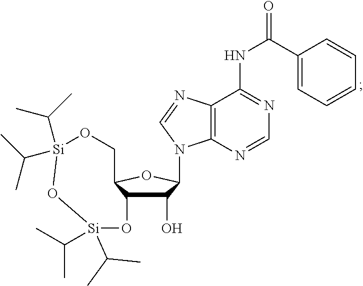

(2) protecting N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide as a methylthiomethyl ether to obtain N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide,

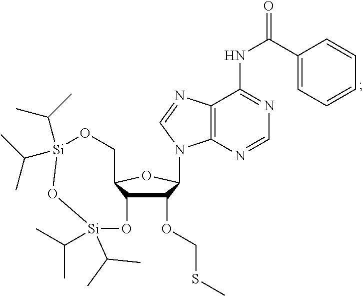

(3) forming a pentaacetate on (2R,3R,4R,5R)-2-amino-3,4,5,6-tetrahydroxyhexanal hydrochloride to obtain (2R,3R,4R,5R,6R)-3-acetamido-6-(acetoxymethyl)-tetrahydro-2H-pyran-2,4,5-triyl triacetate,

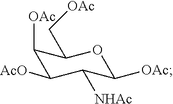 [SMILES: CC(=O)NC1C(OC(C)=O)OC(COC(C)=O)C(OC(C)=O)C1OC(C)=O]

(4) forming a glycosidic bond on (2R,3R,4R,5R,6R)-3-acetamido-6-(acetoxymethyl)-tetrahydro-2H-pyran-2,4,5-triyl triacetate to obtain (2R,3R,4R,5R,6R)-5-acetamido-2-(acetoxymethyl)-6-(hex-5-en-1-yloxy)tetrahydro-2H-pyran-3,4-diyl diacetate,

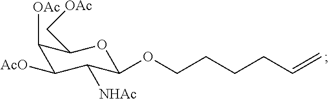 [SMILES: C=CCCCCOC1OC(COC(C)=O)C(OC(C)=O)C(OC(C)=O)C1NC(C)=O]

(5) oxidizing (2R,3R,4R,5R,6R)-5-acetamido-2-(acetoxymethyl)-6-(hex-5-en-1-yloxy)tetrahydro-2H-pyran-3,4-diyl diacetate to obtain 5-(((2R,3R,4R,5R,6R)-3-acetamido-4,5-diacetoxy-6-(acetoxymethyl)-tetrahydro-2H-pyran-2-yl)oxy) pentanoic acid,

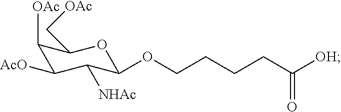 [SMILES: CC(=O)NC1C(OCCCCC(=O)O)OC(COC(C)=O)C(OC(C)=O)C1OC(C)=O]

(6) amidating 5-(((2R,3R,4R,5R,6R)-3-acetamido-4,5-diacetoxy-6-(acetoxymethyl)tetrahydro-2H-pyran-2-yl)oxy) pentanoic acid to obtain (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate,

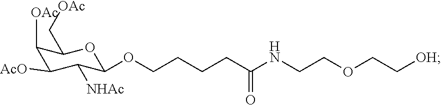 [SMILES: CC(=O)NC1C(OCCCCC(=O)NCCOCCO)OC(COC(C)=O)C(OC(C)=O)C1OC(C)=O]

(7) substituting a sulfoxide on N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide with (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate,

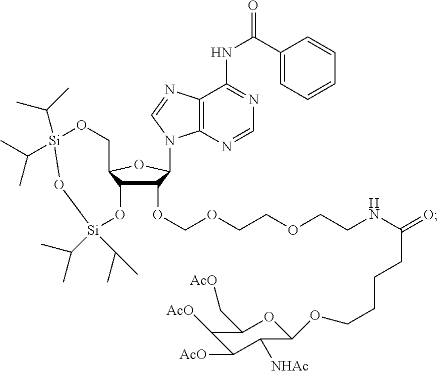

(8) deprotecting (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate,

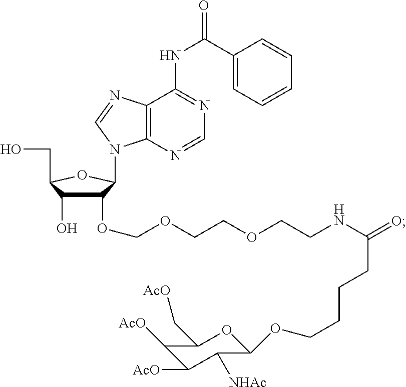

(9) tritylating (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate,

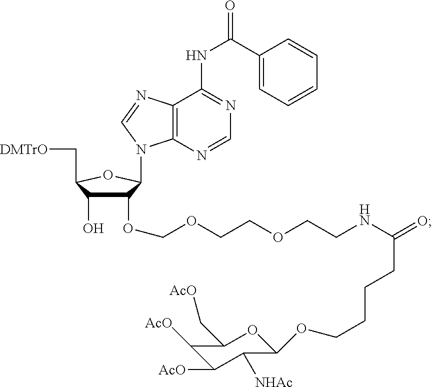

and
(10) phosphorylating (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (adem-A-GalNAc phosphoramidite),

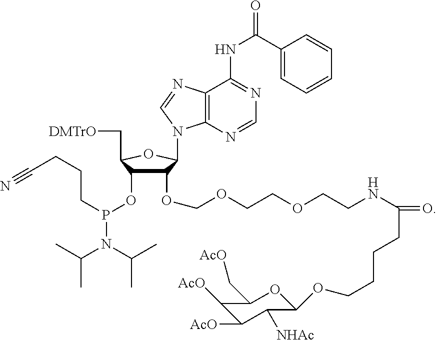

In another instance, adem-A-GalNAc phosphoramidite can be prepared according to the method below, which can include the following steps:
(1′) providing a compound comprising N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide;
(2′) providing a compound comprising (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate;
(3′) substituting a sulfoxide on N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide with (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate;
(4′) deprotecting (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate;
(5′) tritylating (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate; and
(6′) phosphorylating (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (adem-A-GalNAc phosphoramidite).

In yet another instance, adem-A-GalNAc phosphoramidite can be prepared according to the method below, which can include the following steps:
(1′″) substituting a sulfoxide on N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide with (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate,

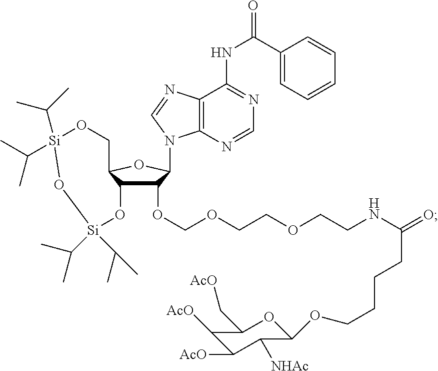

(2′″) deprotecting (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate,

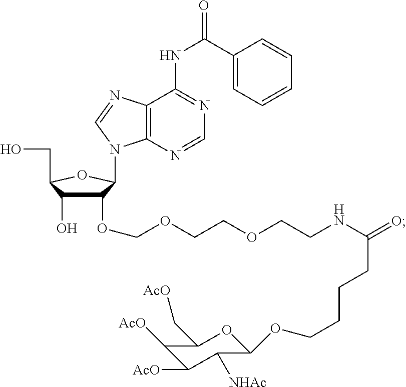

(3′″) tritylating (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate,

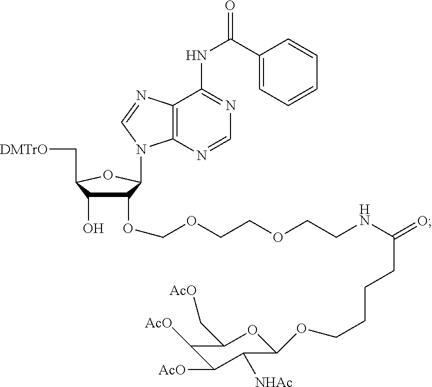

and
(4′″) phosphorylating (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (adem-A-GalNAc phosphoramidite),

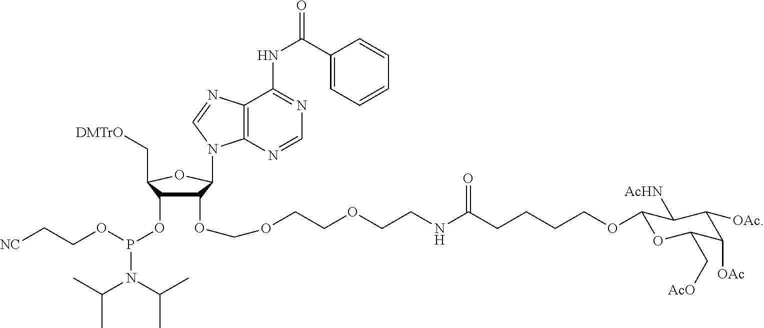

In some instances, N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide can be prepared according to the method below, which can include the following steps:
(1) silylating N6-benzoyladenosine,

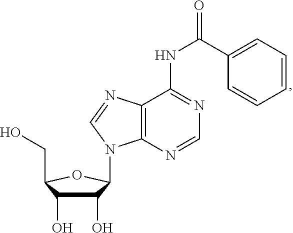

to obtain N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide,

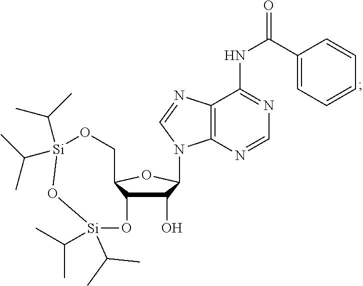

and
(2) protecting N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide as a methylthiomethyl ether to obtain N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide,

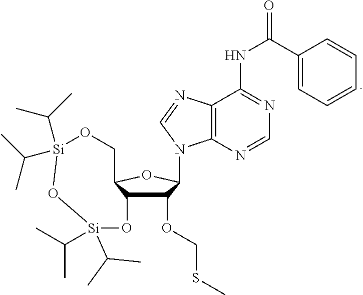

In some instances, (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate can be prepared according to the method below, which can include the following steps:
(1) forming a pentaacetate on (2R,3R,4R,5R)-2-amino-3,4,5,6-tetrahydroxyhexanal hydrochloride to obtain (2S,3R,4R,5R,6R)-3-acetamido-6-(acetoxymethyl)-tetrahydro-2H-pyran-2,4,5-triyl triacetate,

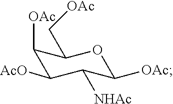 [SMILES: CC(=O)NC1C(OC(C)=O)OC(COC(C)=O)C(OC(C)=O)C1OC(C)=O]

(2) forming a glycosidic bond on (2S,3R,4R,5R,6R)-3-acetamido-6-(acetoxymethyl)-tetrahydro-2H-pyran-2,4,5-triyl triacetate to obtain (2R,3R,4R,5R,6R)-5-acetamido-2-(acetoxymethyl)-6-(hex-5-en-1-yloxy)tetrahydro-2H-pyran-3,4-diyl diacetate,

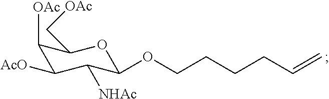 [SMILES: C=CCCCCOC1OC(COC(C)=O)C(OC(C)=O)C(OC(C)=O)C1NC(C)=O]

(3) oxidizing (2R,3R,4R,5R,6R)-5-acetamido-2-(acetoxymethyl)-6-(hex-5-en-1-yloxy)tetrahydro-2H-pyran-3,4-diyl diacetate to obtain 5-(((2R,3R,4R,5R,6R)-3-acetamido-4,5-diacetoxy-6-(acetoxymethyl)-tetrahydro-2H-pyran-2-yl)oxy) pentanoic acid,

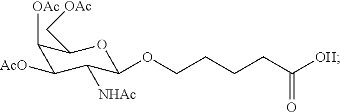 [SMILES: CC(=O)NC1C(OCCCCC(=O)O)OC(COC(C)=O)C(OC(C)=O)C1OC(C)=O]

and
(4) amidating 5-(((2R,3R,4R,5R,6R)-3-acetamido-4,5-diacetoxy-6-(acetoxymethyl)tetrahydro-2H-pyran-2-yl)oxy) pentanoic acid to obtain (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate,

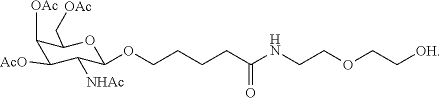 [SMILES: CC(=O)NC1C(OCCCCC(=O)NCCOCCO)OC(COC(C)=O)C(OC(C)=O)C1OC(C)=O]

### EXAMPLES

The following non-limiting example(s) are offered for purposes of illustration, and are not limiting to the scope of the disclosure.

### Example 1: Synthesizing N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide

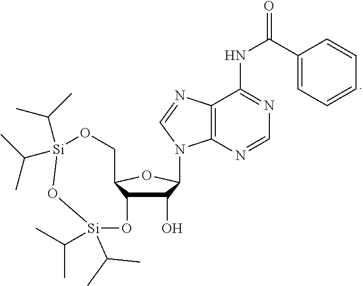

Method: N6-benzoyl adenosine (1 kg, 2.693 mol) was dissolved in dry pyridine. TiPDS-Cl (1.02 kg, 3.23 mol) was added dropwise into the solution while maintaining the temperature at 5° C. The solution was warmed to 25° C., and the mixture was then stirred at 25° C. for 2 hr and was then quenched with MeOH (800 mL). The solution was concentrated under vacuum to remove pyridine, and DCM (10 L) was added. The solution was washed with 30% aqueous citric acid (10 L×2) and 20% aqueous NaCl (10 L), giving a DCM solution (13.3 kg) of the title compound.

Result: Weight: 1.357 kg (as measured by HPLC in the solution); Purity: 92.4%; Yield: 82.1%; ES-MS m/z 614.5 (M+H).

### Example 2: Synthesizing N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide

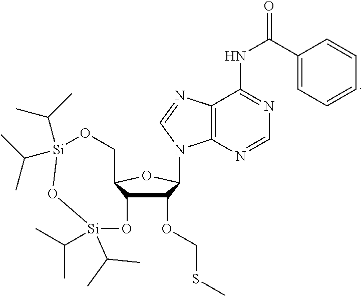

Method: the DCM solution of N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide (13.3 kg) of Example 1 was diluted with DMSO (4.0 L). The solution was concentrated to remove the DCM under vacuum below 45° C. Toluene (6.8 L) was then added to the solution. Acetic acid (4.27 kg) and acetic anhydride (3.1 kg) were sequentially added at 10° C. The reaction was heated to 50° C. and stirred for 42 hr. MeOH (4 L) was added dropwise to quench the reaction at 20° C. The solution then was diluted with EA (14 L) and washed with 25% aqueous K₂CO₃ (14 L×2) and saturated aqueous NaCl (14 L×1). The organic phase was concentrated to 2 V under vacuum and then diluted with n-heptane/EA (2:1; 6.5 L). The solution was filtered through silica gel (7 kg), and the silica gel was eluted with n-heptane/EA (100%˜1/1). The filtrate was concentrated to 1 V to 2 V and then diluted with EA (2.7 L). The resulting solution was added into n-heptane/H₂O (5 V/2 V). The mixture was stirred for 1 hr and then filtered giving the title compound (933 g, 59% purity) as an off-white solid.

Result: ¹H NMR (400 MHz, DMSO-d₆) δ 11.24 (s, 1H), 8.67 (s, 1H), 8.53 (s, 1H), 8.06 (m, 2H), 7.66 (m, 1H), 7.56 (m, 2H), 6.16 (s, 1H), 5.06-4.93 (m, 4H), 4.07-3.97 (m, 4H), 2.11 (s, 1H), 1.24-0.85 (m, 28H); ES-MS m/z 674.5 (M+H).

### Example 3: Synthesizing (2S,3R,4R,5R,6R)-3-acetamido-6-(acetoxymethyl)-tetrahydro-2H-pyran-2,4,5-triyl triacetate

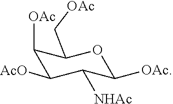 [SMILES: CC(=O)NC1C(OC(C)=O)OC(COC(C)=O)C(OC(C)=O)C1OC(C)=O]

Method: (2R,3R,4R,5R)-2-amino-3,4,5,6-tetrahydroxyhexanal hydrochloride (387 g, 1.79 mol) was dissolved in pyridine (8 V, 3096 mL) under N₂ atmosphere at 20° C. A catalytic amount of DMAP (153 g, 1.26 mol) was added and the mixture was stirred at the same temperature. The mixture was cooled to 0° C. to 10° C., and Ac₂O (6 V, 2322 mL) was added dropwise, and the mixture was stirred at the same temperature. The solution was warmed to 20° C. and stirred for 18 hr. Toluene (12 V) was added to the reaction solution at 20° C., and the mixture was stirred for 30 min at 20° C. The solution was then concentrated, and MeOH (5 V) was added and stirred at 20° C. for 2 hr. The solids were filtered, and the resulting cake was washed with MeOH. The solid was then dried under vacuum to give the title compound (600 g, 86%) as a white solid.

Result: Weight: ¹H NMR (400 MHz, DMSO-d₆) δ 7.89 (d, J=9.3 Hz, 1H), 5.64 (d, J=8.8 Hz, 1H), 5.29-5.24 (m, 1H), 5.06 (dd, J=11.4, 3.4 Hz, 1H), 4.26-4.17 (m, 1H), 4.13-3.96 (m, 3H), 2.12 (s, 3H), 2.03 (s, 3H), 1.99 (s, 3H), 1.90 (s, 3H), 1.78 (s, 3H); ES-MS m/z 406.8 (M+NH₄).

### Example 4: Synthesizing (2R,3R,4R,5R,6R)-5-acetamido-2-(acetoxymethyl)-6-(hex-5-en-1-yloxy)tetrahydro-2H-pyran-3,4-diyl diacetate

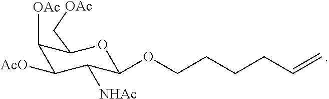 [SMILES: C=CCCCCOC1OC(COC(C)=O)C(OC(C)=O)C(OC(C)=O)C1NC(C)=O]

Method: (2S,3R,4R,5R,6R)-3-acetamido-6-(acetoxymethyl)-tetrahydro-2H-pyran-2,4,5-triyl triacetate (150 g, 0.39 mol) of Example 3 was dissolved in DCM (10 V, 1.5 L). 5-hexenol (44.34 g, 1.15 eq, 44.9 mol) was added to the mixture, and the mixture was stirred for 10 min to 15 min at 20° C. FeCl₃ (37.4 g, 0.6 eq, 37.95 mol) was added to the mixture at 20° C. under N₂ atmosphere. The mixture was then stirred at 20° C. until complete conversion (i.e., about 16 hr). Once the reaction was deemed complete, H₂O (5 V) was added, and the mixture was stirred for 30 min. The mixture was extracted with DCM (2×10 V). The organic layers were combined, dried over Na₂SO₄, filtered and concentrated to 1 V. n-Heptane (10 V to 15 V) was added at 20° C., and the mixture was stirred for 4 hr. The solid was filtered and dried to give the title compound (149 g, 90%) as a white solid.

Result: ¹H NMR (400 MHz, DMSO-d₆) δ 7.80 (d, J=9.1 Hz, 1H), 5.75 (s, 2H), 5.24-5.16 (m, 1H), 5.03-4.91 (m, 3H), 4.49 (d, J=8.5 Hz, 1H), 4.02 (s, 3H), 3.92-3.81 (m, 1H), 3.79-3.63 (m, 1H), 3.49-3.36 (m, 1H), 2.10 (s, 3H), 2.05-1.95 (m, 6H), 1.89 (s, 3H), 1.76 (s, 3H), 1.52-1.43 (m, 3H), 1.41-1.32 (m, 2H); ES-MS m/z 430 (M+H).

### Example 5: Synthesizing 5-(((2R,3R,4R,5R,6R)-3-acetamido-4,5-diacetoxy-6-(acetoxymethyl)tetrahydro-2H-pyran-2-yl)oxy) pentanoic acid

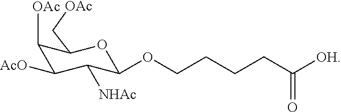 [SMILES: CC(=O)NC1C(OCCCCC(=O)O)OC(COC(C)=O)C(OC(C)=O)C1OC(C)=O]

Method: ((2R,3R,4R,5R,6R)-5-acetamido-2-(acetoxymethyl)-6-(hex-5-en-1-yloxy)tetrahydro-2H-pyran-3,4-diyl diacetate (143 g, 0.33 mol) of Example 4 was dissolved in DCM: CH₃CN: H₂O (3.5 V: 3.5 V: 4.75 V) at 20° C., and the mixture was stirred for 20 min. The mixture was cooled to 0° C. to 5° C., and NaIO₄ (284 g, 4 eq, 1.32 mol) was added followed by RuCl₃·H₂O (1.723 g, 0.027 eq, 0.00891 mol). The reaction mixture was stirred at 5° C. to 20° C., and an additional amount of NaIO₄ (2 eq, 0.66 mol) was added and the mixture was stirred for an additional 2 hr. The periodate salt was filtered with DCM (2 V). The organic layer was separated, and the aqueous layer was extracted back by DCM (2×10 V) by saturating the aqueous layer with NaCl. The organic layers were combined and concentrated to 1 V to 2 V. MTBE was added dropwise (10 V to 15 V) with constant stirring over 2 hr to 4 hr. The solids were filtered, and the cake was washed with MTBE (2 V). The solid was dried to give the title compound (140 g, 79%) as white solid.

Result: ¹H NMR (400 MHz, DMSO-d₆) δ=11.98 (br s, 1H), 7.80 (d, J=9.3 Hz, 1H), 5.21 (d, J=3.4 Hz, 1H), 4.96 (dd, J=11.3, 3.4 Hz, 1H), 4.49 (d, J=8.4 Hz, 1H), 4.09-3.96 (m, 3H), 3.87 (td, J=11.1, 8.9 Hz, 1H), 3.81-3.61 (m, 1H), 3.47-3.38 (m, 1H), 2.20 (s, 2H), 2.10 (s, 3H), 2.00 (s, 4H), 1.89 (s, 3H), 1.77 (s, 3H), 1.55-1.43 (m, 5H); ES-MS m/z 448 (M+H).

### Example 6: Synthesizing (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate

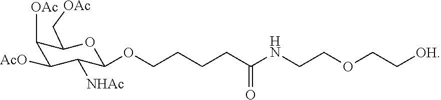 [SMILES: CC(=O)NC1C(OCCCCC(=O)NCCOCCO)OC(COC(C)=O)C(OC(C)=O)C1OC(C)=O]

Method: 5-(((2R,3R,4R,5R,6R)-3-acetamido-4,5-diacetoxy-6-(acetoxymethyl)tetrahydro-2H-pyran-2-yl)oxy) pentanoic acid (470 g, 1.05 mol) of Example 5 was dissolved in DCM (4.7 L). CDI (205 g, 1.26 mol) was added at 5° C., and the mixture was stirred for 2 hr at 20° C. The resulting mixture was added to a solution of 2-(2-aminoethoxy) ethan-1-ol (132.5 g, 1.26 mol) in DCM (940 mL) at 5° C. The reaction mixture was warmed to 20° C. and was stirred for 2 hr. Tartaric acid (379 g, 2.4 eq, 2.52 mol) in THF (3.76 L) was added to the reaction solution at 5° C. The mixture was stirred for 1 hr at 5° C. and then filtered. The filtrate was concentrated and diluted with DCM (4 V), then filtered through silica gel (2.35 kg). The silica gel was eluted with a gradient of 100% DCM to DCM/MeOH (20/1), and fractions containing the compound were collected and combined. The solution was concentrated and diluted with THF to 10 V to give 4.0 kg THF solution of the title compound.

Result: Weight: 468 g (as determined by HPLC of the THF solution); Purity: 98.3%; Yield 83.3%; ES-MS m/z 535.4 (M+H).

### Example 7: Synthesizing (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate

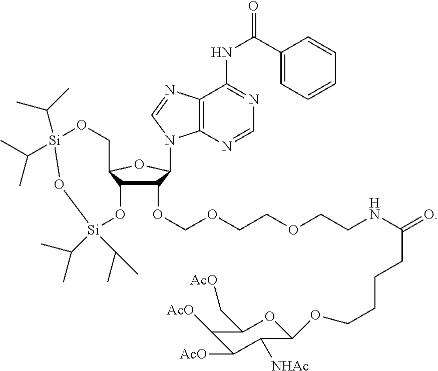

Method: N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide (508.8 g, Assay: 93.6%; 0.71 mol) was added to the THF solution of (((2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate (3.874 kg, Assay: 11.7%; 0.85 mol) of Example 6. The solution was concentrated to remove H₂O, and then diluted with THF (9.5 L). 4 Å molecular sieves (476 g) were added, and the mixture was stirred for 30 min at 20° C. NIS (191 g, 1.2 eq, 0.852 mol) and TfOH (213 g, 2.0 eq, 1.42 mol) were sequentially added at −35° C. to −20° C., and the mixture was stirred at that temperature for 2 hr, and then at 5° C. for another 3 hr. The solution was cooled to −35° C. to −20° C., and the reaction was quenched with TEA (210 g) at −35° C. to −20° C. The reaction mixture was filtered, and the filtrate was washed with 10% aqueous Na₂SO₃ (4.8 L) and saturated aqueous NaCl (4.8 L) to give a 10.05 kg THF solution of the title compound.

Result: Weight: 763.8 g (as determined by HPLC); Purity: 86.7%; Yield: 93.2%; ES-MS m/z 1160.91 (M+H).

### Example 8: Synthesizing (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate

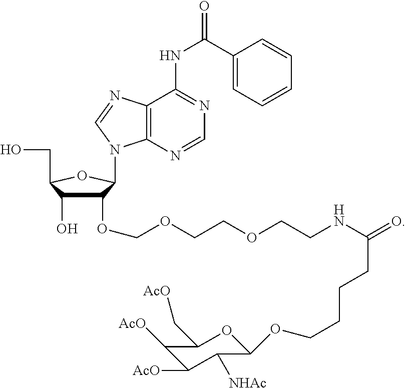

Method: A THF solution of (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate (9.9 kg, Assay: 7.6%; 0.65 mol) of Example 7 was concentrated to remove H₂O and then diluted to 7.5 L with THF. TEA (197 g, 3.0 eq, 1.95 mol) and TEA. 3HF (314 g, 3.0 eq, 1.95 mol) were added at 5° C., and the mixture was stirred for 12 hr at that temperature. DCM (7.5 L) was added, and the mixture was concentrated to 5V to 6 V under vacuum. DCM (7.5 L×2) was added again. The solution was sequentially washed with 7% aqueous NaHCO₃ (7.5 L) and saturated aqueous NaCl (7.5 L) to give a 12.5 kg DCM solution of the title compound.

Result: Weight: 550 g (as determined by quantitative HPLC); Purity: 86.5%; Yield: 92.4%; ES-MS m/z 918.6 (M+H).

### Example 9: Synthesizing (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate

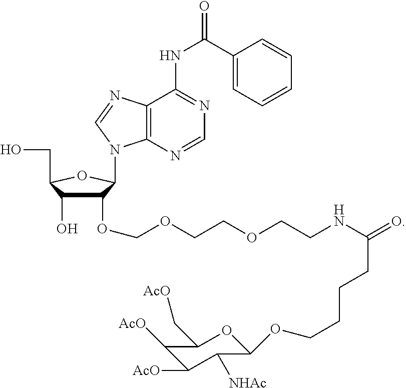

Method: A DCM solution of (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate (1.35 kg, HPCL Assay: 4.4%; 0.54 mol) of Example 8 was concentrated to 5 V to 6 V. DCM (5 L) was then added to dilute the solution. 4 Å molecular sieves (500 g) were added, and the mixture was stirred for 30 min at 5° C. NMM (221 g, 4.0 eq, 4.16 mol) and DMTr-Cl (203 g, 1.1 eq, 0.594 mol); dissolved in 1.5 L DCM) were sequentially added, and the mixture was stirred at 5° C. for 2 hr. The mixture was filtered, and the solution is washed with 7% aqueous NaHCO₃ (2.5 L) and water (2.5 L×2). The DCM solution was concentrated to 2 V to 3 V, and EA (5 L) was added. The solution was concentrated to 3 V to 4 V, and then n-heptane/MTBE (12.5 L/12.5 L) was added. The resulting mixture was purified by chromatography on silica gel (4 kg) using a gradient of 100% EA to 1/1 EA/acetone. The appropriate fractions are concentrated to an oil. A solution of EA+0.5% TEA (5 L) was added, and the mixture was concentrated to 1.5 L. The EA solution was added to n-heptane/MTBE (5.45 L/1.09 L+0.5% TEA) at 5° C. The mixture was stirred at 5° C.±5° C. for 1 hr and then filtered, and the wet cake was washed with n-heptane (IL). The wet cake was purified by chromatography on silica gel (4.35 kg) again using a gradient of 100% EA to 1/1 EA/acetone. The appropriate fractions were concentrated to oil. EA+0.5% TEA (1.08 L) was added, and the EA solution was added to n-heptane/MTBE (4.5 L/0.9 L+0.5% TEA) at 5° C. The mixture was stirred at 5° C. for 1 hr and then filtered, and the wet cake was washed with n-heptane (1 L) and dried under vacuum at 45° C. to give the title compound (381 g, 55%).

Results: ¹H NMR (400 MHz, CDCl₃) δ 9.01 (s, 1H), 8.63 (s, 1H), 8 8.18 (s, 1H), 8 7.98 (d, 2H), 8 7.57-7.53 (m, 1H), 7.48-7.44 (m, 2H), 7.38-7.31 (m, 2H), 7.21-7.10 (m, 8H), 6.79-6.71 (m, 5H), 6.39 (d, 1H), 6.2 (d, 1H), 5.3-5.24 (m, 1H), 5.15 (dd, 1H), 4.94-4.88 (m, 1H), 4.76 (2, 2H), 4.58 (d, 1H), 4.49-4.44 (m, 1H), 4.29-4.22 (m, 1H), 4.11-3.92 (m, 4H), 3.87-3.75 (m, 3H), 3.71 (s, 6H), 3.67-3.30 (m, 10H), 2.06 (s, 3H), 1.95 (s, 3H), 1.91 (s, 3H), 1.89-1.86 (m, 2H), 1.85 (s, 3H), 1.62-1.4 (m, 4H); ES-MS m/z 1220.2 (M+H).

### Example 10: Synthesizing (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (adem-A-GalNAc phosphoramidite)

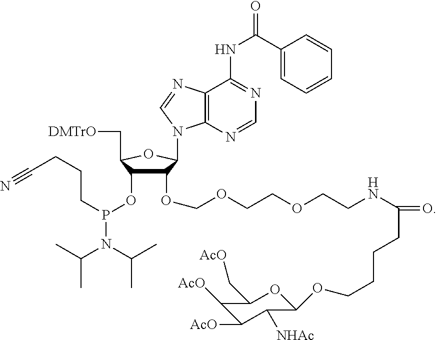

Method: (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate (246 g, HPLC Assay: 94.9%; 0.19 mol) of Example 9 was added to a solution of NMI (16.0 g, 1.0 eq, 0.19 mol) and DCM (2.46 L). The solution was concentrated to 5 V, and DCM (1.23 L) was added. The solution was then cooled to 5° C., and 4 Å molecular sieves (234 g) and tetrazole (6.7 g, 0.5 eq, 0.95 mol) were sequentially added. The solution was purged with N₂ and bis(diisopropylamino)-phosphanyloxypropanenitrile (92.3 g, 1.6 eq, 0.304 mol) was added at 5° C. The mixture was warmed to 20° C. and then stirred at that temperature for 4 hr. The mixture was filtered, and the solution was washed with 7% aqueous NaHCO₃ (1.17 L×1), water (1.87 L×1) and saturated aqueous NaCl (1.17 L×2). The DCM solution was concentrated to an oil, and EA (3.5 L)/MTBE (7.0 L) is added. The solution was washed with DMF/H₂O (1/1, 3.5 L×2), saturated aqueous NaCl (3.5 L), DMF/H₂O (1/1, 3.5 L×2) and saturated aqueous NaCl (3.5 L×2). TEA (55 mL, 0.5% in MTBE, 0.39 mol) was charged, and the resulting solution was dried over Na₂SO₄ (750 g) for 1 hr. The mixture was filtered and concentrated to 5 V. The solution was added into n-heptane/MTBE (3/2, 0.96 L/0.64 L+0.5% TEA) at 5° C., and the mixture was stirred for 1 hr at that temperature. The mixture was filtered, and the filter cake was washed with n-heptane/MTBE (3/2, 0.3 L/0.2 L+0.5% TEA). The solids were dissolved in EA+0.5% TEA (0.2 L), and the solution was added to n-heptane/MTBE (3/2, 0.96 L/0.64 L+0.5% TEA) at 5° C. The mixture was filtered, and the solids were washed with n-heptane/MTBE (3/2, 50 mL/33 mL+0.5% TEA). The solids were dried at 50° C. under vacuum for 61 hr to give the title compound (289.55 g; Yield: 100%) as a white solid.

Results: ¹H NMR (400 MHz, DMSO-d₆) δ 11.26 (s, 1H), 8.71-8.60 (m, 2H), 7.82 (d, 1H), 7.79-7.72 (m, 1H), 7.69-7.61 (m, 1H), 7.59-7.53 (m, 2H), 7.43-7.35 (m, 2H), 7.32-7.16 (m, 7H), 6.90-6.76 (m, 4H), 6.29-6.20 (m, 1H), 5.28-5.16 (m, 2H), 5.01 (dd, 1H), 4.83-4.70 (m, 3H), 4.52 (d, 1H), 4.36-4.24 (m, 1H), 4.11-3.99 (m, 3H), 3.95-3.80 (m, 2H), 3.78-3.66 (m, 8H), 3.65-3.53 (m, 2H), 3.5-3.37 (m, 3H), 3.34-3.19 (m, 5H), 3.13-3.05 (m, 3H), 2.82-2.66 (m, 2H), 2.18 (s, 3H), 2.06-2.03 (m, 2H), 2.00 (s, 3H), 1.91 (s, 3H), 1.78 (s, 3H), 1.54-1.39 (m, 4H), 1.29-1.20 (m, 2H), 1.19-1.12 (m, 9H), 1.11-1.09 (m, 4H), 1.07-1.03 (m, 2H). 31P-NMR (400 MHz, DMSO-d₆) δ 149.36. HRMS (ESI) calculated for C₇₁H₉₀N₉O₂₀P (M+H)⁺1420.6112, found 1420.6083/1420.6093.

1. A method of making a compound represented by a structure of: 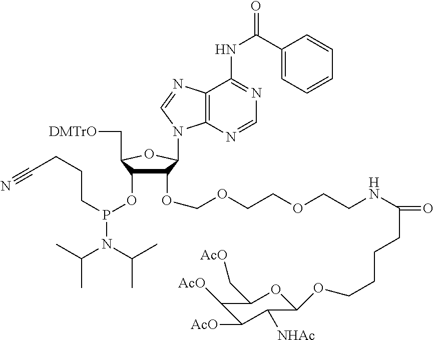
(adem-A-GalNAc phosphoramidite),
the method comprising the steps of:
silylating N6-benzoyladenosine to obtain N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide;
protecting N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide as a methylthiomethyl ether to obtain N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide;
forming a pentaacetate on (2R,3R,4R,5R)-2-amino-3,4,5,6-tetrahydroxyhexanal hydrochloride to obtain (2R,3R,4R,5R,6R)-3-acetamido-6-(acetoxymethyl)-tetrahydro-2H-pyran-2,4,5-triyl triacetate;
forming a glycosidic bond on (2R,3R,4R,5R,6R)-3-acetamido-6-(acetoxymethyl)-tetrahydro-2H-pyran-2,4,5-triyl triacetate to obtain (2R,3R,4R,5R,6R)-5-acetamido-2-(acetoxymethyl)-6-(hex-5-en-1-yloxy)tetrahydro-2H-pyran-3,4-diyl diacetate;
oxidizing (2R,3R,4R,5R,6R)-5-acetamido-2-(acetoxymethyl)-6-(hex-5-en-1-yloxy)tetrahydro-2H-pyran-3,4-diyl diacetate to obtain 5-(((2R,3R,4R,5R,6R)-3-acetamido-4,5-diacetoxy-6-(acetoxymethyl)-tetrahydro-2H-pyran-2-yl)oxy) pentanoic acid;
amidating 5-(((2R,3R,4R,5R,6R)-3-acetamido-4,5-diacetoxy-6-(acetoxymethyl)tetrahydro-2H-pyran-2-yl)oxy) pentanoic acid to obtain (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate;
substituting a sulfoxide on N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide with (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate;
deprotecting (2R,3R,4R,5R,6R)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate;
tritylating (2R,3R,4R,5R,6R)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate; and
phosphorylating (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate to obtain (2R,3R,4R,5R,6R)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (adem-A-GalNAc phosphoramidite).

2. method of making a compound represented by a structure of: 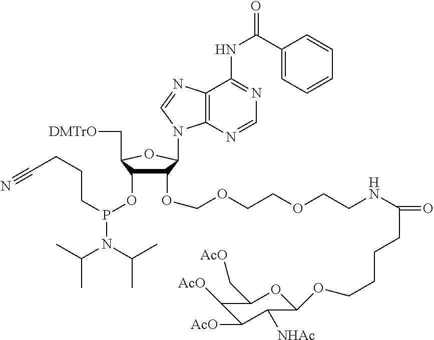
(adem-A-GalNAc phosphoramidite),
the method comprising the step of:
providing a compound comprising N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide;
providing a compound comprising (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate;
substituting a sulfoxide on N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide with (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate to obtain (2S,3S,4S,5S,6S)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate;
deprotecting (2S,3S,4S,5S,6S)-5-acetamido-2-(acetoxymethyl)-6-((5-((2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy)ethyl)amino)-5-oxopentyl)oxy)tetrahydro-2H-pyran-3,4-diyl diacetate to obtain (2S,3S,4S,5S,6S)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate;
tritylating (2S,3S,4S,5S,6S)-5-acetamido-2-(acetoxymethyl)-6-((5-((2-(2-((((2R,3R,4R,5R)-2-(6-benzamido-9H-purin-9-yl)-4-hydroxy-5-(hydroxymethyl)tetrahydrofuran-3-yl)oxy)methoxy)ethoxy)ethyl)amino)-5-oxopentyl)oxy)tetrahydro-2H-pyran-3,4-diyl diacetate to obtain (2S,3S,4S,5S,6S)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate; and
phosphorylating (2S,3S,4S,5S,6S)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-hydroxytetrahydrofuran-3-yl)oxy)methoxy)ethoxy)ethyl)amino)-5-oxopentyl)oxy)tetrahydro-2H-pyran-3,4-diyl diacetate to obtain (2S,3S,4S,5S,6S)-5-acetamido-2-(acetoxymethyl)-6-((5-((2-(2-((((2R,3R,4R,5R)-2-(6-benzamido-9H-purin-9-yl)-5-((bis(4-methoxyphenyl)(phenyl)methoxy)methyl)-4-(((2-cyanoethoxy)(diisopropylamino)phosphaneyl)oxy)tetrahydrofuran-3-yl)oxy)methoxy)ethoxy)ethyl)amino)-5-oxopentyl)oxy)tetrahydro-2H-pyran-3,4-diyl diacetate (adem-A-GalNAc phosphoramidite).

3. The method of claim 2, wherein N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide is prepared by a method comprising:
silylating N6-benzoyladenosine to obtain N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide; and
protecting N-(9-((6aR,8R,9R,9aS)-9-hydroxy-2,2,4,4-tetraisopropyltetrahydro-6H-furo[3,2-f][1,3,5,2,4]trioxadisilocin-8-yl)-9H-purin-6-yl)benzamide as a methylthiomethyl ether to obtain N-(9-((6aR,8R,9R,9aR)-2,2,4,4-tetraisopropyl-9-((methylthio)methoxy)tetrahydro-6H-furo[3,2-f][1,3,5,2,4]trioxadisilocin-8-yl)-9H-purin-6-yl)benzamide.

4. The method of claim 2, wherein (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate is prepared by a method comprising:
forming a pentaacetate on (2R,3R,4R,5R)-2-amino-3,4,5,6-tetrahydroxyhexanal hydrochloride to obtain (2S,3R,4R,5R,6R)-3-acetamido-6-(acetoxymethyl)-tetrahydro-2H-pyran-2,4,5-triyl triacetate;
forming a glycosidic bond on (2S,3R,4R,5R,6R)-3-acetamido-6-(acetoxymethyl)-tetrahydro-2H-pyran-2,4,5-triyl triacetate to obtain (2R,3R,4R,5R,6R)-5-acetamido-2-(acetoxymethyl)-6-(hex-5-en-1-yloxy)tetrahydro-2H-pyran-3,4-diyl diacetate;
oxidizing (2R,3R,4R,5R,6R)-5-acetamido-2-(acetoxymethyl)-6-(hex-5-en-1-yloxy)tetrahydro-2H-pyran-3,4-diyl diacetate to obtain 5-(((2R,3R,4R,5R,6R)-3-acetamido-4,5-diacetoxy-6-(acetoxymethyl)-tetrahydro-2H-pyran-2-yl)oxy) pentanoic acid; and
amidating 5-(((2R,3R,4R,5R,6R)-3-acetamido-4,5-diacetoxy-6-(acetoxymethyl)tetrahydro-2H-pyran-2-yl)oxy) pentanoic acid to obtain (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate.

5. The method of claim 3, wherein (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate is prepared by a method comprising:
forming a pentaacetate on (2R,3R,4R,5R)-2-amino-3,4,5,6-tetrahydroxyhexanal hydrochloride to obtain (2S,3R,4R,5R,6R)-3-acetamido-6-(acetoxymethyl)-tetrahydro-2H-pyran-2,4,5-triyl triacetate;
forming a glycosidic bond on (2S,3R,4R,5R,6R)-3-acetamido-6-(acetoxymethyl)-tetrahydro-2H-pyran-2,4,5-triyl triacetate to obtain (2R,3R,4R,5R,6R)-5-acetamido-2-(acetoxymethyl)-6-(hex-5-en-1-yloxy)tetrahydro-2H-pyran-3,4-diyl diacetate;
oxidizing (2R,3R,4R,5R,6R)-5-acetamido-2-(acetoxymethyl)-6-(hex-5-en-1-yloxy)tetrahydro-2H-pyran-3,4-diyl diacetate to obtain 5-(((2R,3R,4R,5R,6R)-3-acetamido-4,5-diacetoxy-6-(acetoxymethyl)-tetrahydro-2H-pyran-2-yl)oxy) pentanoic acid; and
amidating 5-(((2R,3R,4R,5R,6R)-3-acetamido-4,5-diacetoxy-6-(acetoxymethyl)tetrahydro-2H-pyran-2-yl)oxy) pentanoic acid to obtain (2R,3R,4R,5R,6R)-5-acetamido-2-(acetoxymethyl)-6-((5-((2-(2-hydroxyethoxy)ethyl)amino)-5-oxopentyl)oxy)tetrahydro-2H-pyran-3,4-diyl diacetate.
